(19) United States
(12) Patent Application Publication
(10) Pub. No.: US 2016/0158576 A1
(43) Pub. Date: Jun. 9, 2016

(54) Two-And Three-Dimensional Far Infrared Acupuncture Device For Therapy And Rehabilitation

(71) Applicants: Chao-Kuo Tseng, New Taipei City (TW); Hsiao-Chien Tseng, New Taipei City (TW); Hsiao-Yun Tseng, New Taipei City (TW)

(72) Inventors: Chao-Kuo Tseng, New Taipei City (TW); Hsiao-Chien Tseng, New Taipei City (TW); Hsiao-Yun Tseng, New Taipei City (TW)

(21) Appl. No.: 14/558,738

(22) Filed: Dec. 3, 2014

(51) Int. Cl. A61N 5/06; A61N 2/00

(52) U.S. Cl. CPC A61N 5/0619; A61N 2/002; A61N 2/004; A61N 2005/066; A61N 2005/0626

(57) ABSTRACT

Disclosed is a two- and three-dimensional far infrared acupuncture device for therapy and rehabilitation, which includes: at least one tubular body including a far infrared ceramic material and having two ends respectively forming an opening and a ventilation hole and including an accommodation space formed between the opening and the ventilation hole; and at least one electrical heating unit having an end forming a handgrip section that includes a control device mounted thereto. The electrical heating unit has an end opposite to the handgrip section and insertable through the opening into the tubular body. The control device controls the temperature of the electrical heating unit in a range of 37-50 degrees Celsius so that the temperature is transmitted to the tubular body so that the tubular body emits far infrared light having a wavelength of 4-14 micrometers to stimulate the acupoints and organs of the human body.

### BACKGROUND OF THE INVENTION

(a) Technical Field of the Invention

The present invention generally relates to a therapy and rehabilitation device, and more particularly to a two- and three-dimensional far infrared acupuncture device for therapy and rehabilitation that makes use of heat, far infrared light, and a bar-like structure to stimulate acupuncture points of human body, as well as organs including skin, blood vessels, muscles, and viscera, to achieve effects of therapy and rehabilitation.

(b) Description of the Prior Art

Acupuncture has been used in Chinese medicine system for thousands of years. The acupuncture applies stimulation to acupuncture points of a human body having a diameter around 0.5-0.8 mm and uses related acupuncture meridian and nerve system to treat diseases. However, the acupuncture points of the acupuncture meridian are of various depths under the skin of around 1-3 cuns (a traditional Chinese unit of length). It is an ancient saying that it is as deep as a well in the abdomen and as thin as a biscuit in the back; no deep penetration is allowed in the area from the neck to medulla oblongata; bladder meridian is on the back side of the lower leg; and Weizhong acupoint to Chengshan acupoint extends for 4-5 cuns.

Taking Zusanli acupoint of stomach meridian of foot-yangming as an example, needle penetration of 1 cun at a location that is below outer Xiyang acupoint by 3 cuns is used to treat gynaecological diseases, needle penetration of 2 cuns is used to treat asthma, and needle penetration of 3 cuns is used to treat heart diseases. In addition, needle penetration may be classified according to thickness of acupoint, fatness of body, severity of disease, chronicity of disease, operation time of acupuncture meridian system, and depth of acupuncture meridian and internal organs.

The above is made based on the example of Zusanli acupoint and there are 360 important acupoints in a human body. To make a correct penetration of needle, no scientific way is known for making precise penetration of needle and it can only rely on acupuncture masters' experiences. It basically needs fortune to have effective therapy. Further, general people have no acupuncture knowledge regarding acupoints and depth issue and it is often risky for general people to receive incorrect acupuncture therapy and may even making the disease worse.

Further, although the traditional acupuncture provides an effect of therapy, needle penetration is still considered an invasive procedure and severe standard of disinfection is required to avoid undesired transmission of other diseases.

In view of these problems, the present invention aims to provide a solution to overcome these problems.

### SUMMARY OF THE INVENTION

In view of the above-discussed drawbacks, the present invention provides a two- and three-dimensional far infrared acupuncture device for therapy and rehabilitation, which comprises: at least one tubular body, which comprises a far infrared ceramic material, the tubular body having two ends respectively forming an opening and a ventilation hole and comprising an accommodation space between the opening and the ventilation hole; and at least one electrical heating unit, which has an end forming a handgrip section, the handgrip section comprising a control device mounted thereto. The electrical heating unit has an end opposite to the handgrip section and insertable through the opening into the tubular body. The control device controls the temperature of the electrical heating unit within the range of 37-50 degrees Celsius so that the temperature is transmitted to the tubular body. The tubular body emits far infrared light having a wavelength of 4-14 micrometers to stimulate the acupoints and organs of the human body for achieving an effect of therapy.

The primary object of the present invention is to make the operation easy and to use heat and far infrared light to simulate acupoints and organs of a human body. Further, a bar-like design is adopted, allowing the far infrared light to deeply penetrate into the human body so as to throughout improve the invasive rehabilitation and therapy processes that are used conventionally to achieved enhanced security and sanitary. Further, the present invention can be mounted to a chair and may include a connection enclosure unit on which markings are provided to make recording of the treated portion of a user easy and convenient for subsequent use thereby eliminating the troubles of setting and improving the convenience of use.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a schematic view, partly broken, showing a two- and three-dimensional far infrared acupuncture device for therapy and rehabilitation according to a first embodiment of the present invention.

FIG. 2 is an exploded view showing the two- and three-dimensional far infrared acupuncture device for therapy and rehabilitation according to the first embodiment of the present invention.

FIG. 3 is a schematic view illustrating a condition of use of the two- and three-dimensional far infrared acupuncture device for therapy and rehabilitation according to the first embodiment of the present invention.

FIG. 4 is a schematic view illustrating a condition of use of the two- and three-dimensional far infrared acupuncture device for therapy and rehabilitation according to the first embodiment of the present invention.

FIG. 5 is a schematic view, partly broken, showing a two- and three-dimensional far infrared acupuncture device for therapy and rehabilitation according to a second embodiment of the present invention.

FIG. 6 is a schematic view illustrating a condition of use of FIG. 5.

FIG. 7 is a schematic view, partly broken, showing a two- and three-dimensional far infrared acupuncture device for therapy and rehabilitation according to a third embodiment of the present invention.

FIG. 8 is a schematic view illustrating a condition of use of FIG. 7.

FIG. 9 is a cross-sectional view illustrating a condition of use of FIG. 7.

FIG. 10 is a schematic view illustrating a magnetic field of FIG. 9.

FIG. 11 is a perspective view showing a two- and three-dimensional far infrared acupuncture device for therapy and rehabilitation according to a fourth embodiment of the present invention.

FIG. 12 is a schematic view illustrating a condition of use of FIG. 10.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

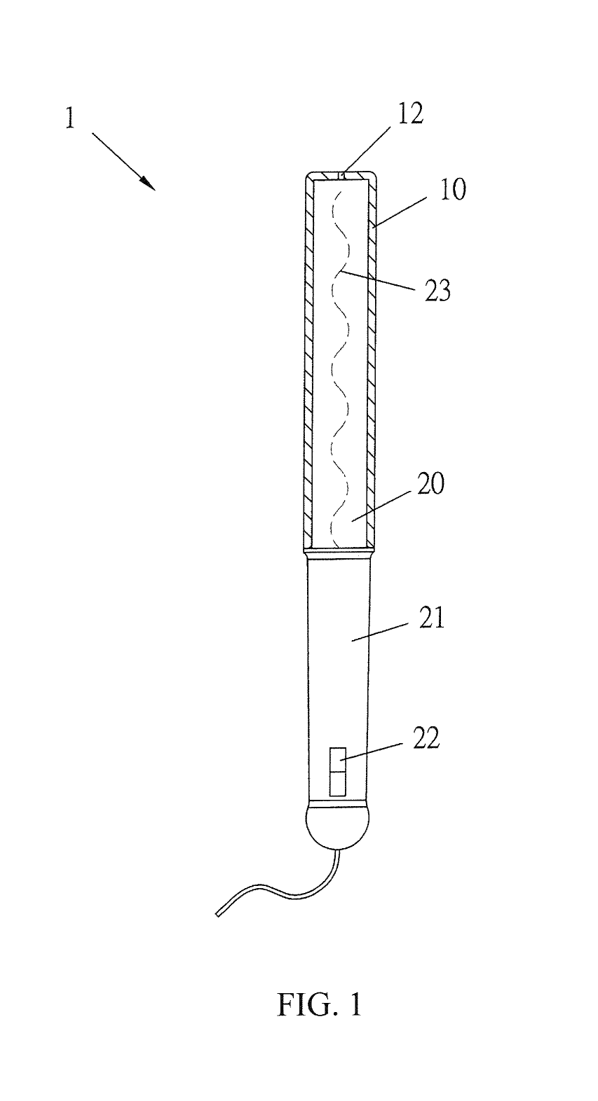

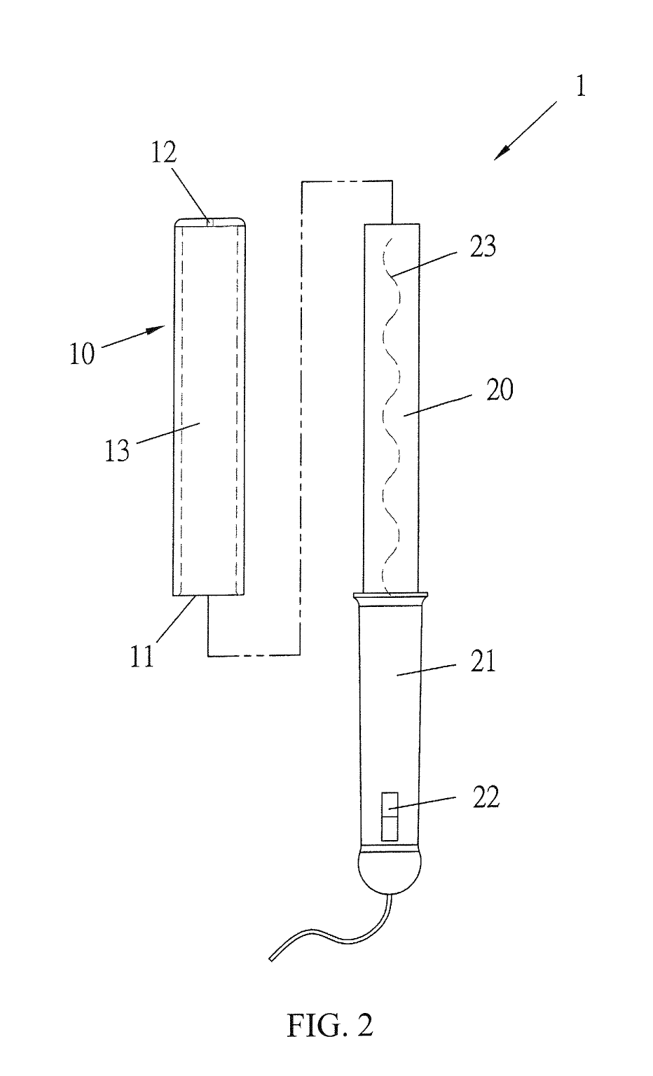

Firstly, referring to FIGS. 1 and 2, a two- and three-dimensional far infrared acupuncture device for therapy and rehabilitation according to a first embodiment of the present invention, generally designated at 1, comprises: at least one tubular body 10 and at least one electrical heating unit 20.

The tubular body 10 comprises a far infrared ceramic material. The tubular body 10 has two ends respectively forming an opening 11 and a ventilation hole 12 and also comprises an accommodation space 13 formed between the opening 11 and the ventilation hole 12.

The electrical heating unit 20 has an end that comprises a handgrip section 21. The handgrip section 21 comprises a control device 22 mounted thereto. The electrical heating unit 20 has an end that is opposite to the handgrip section 21 and is insertable through the opening 11 of the tubular body 10 into the accommodation space 13 of the tubular body 10. The control device 22 controls the temperature of the electrical heating unit 20 within the range of 37-50 degrees Celsius so that the temperature is transmitted to the tubular body 10.

The electrical heating unit 20 comprises at least one nichrome wire 23 arranged therein and electrically connected with the control device 22.

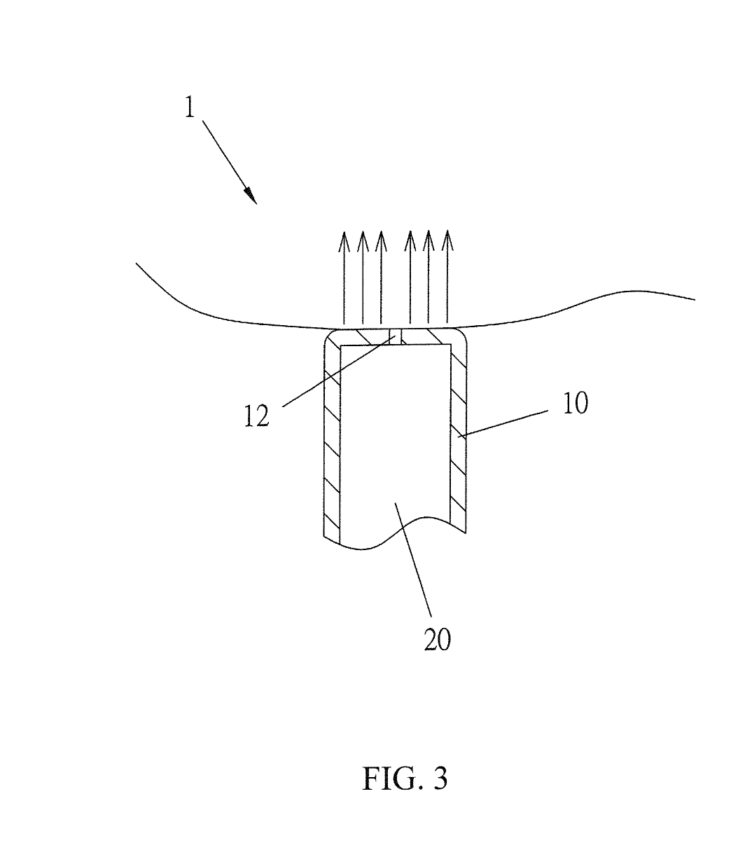

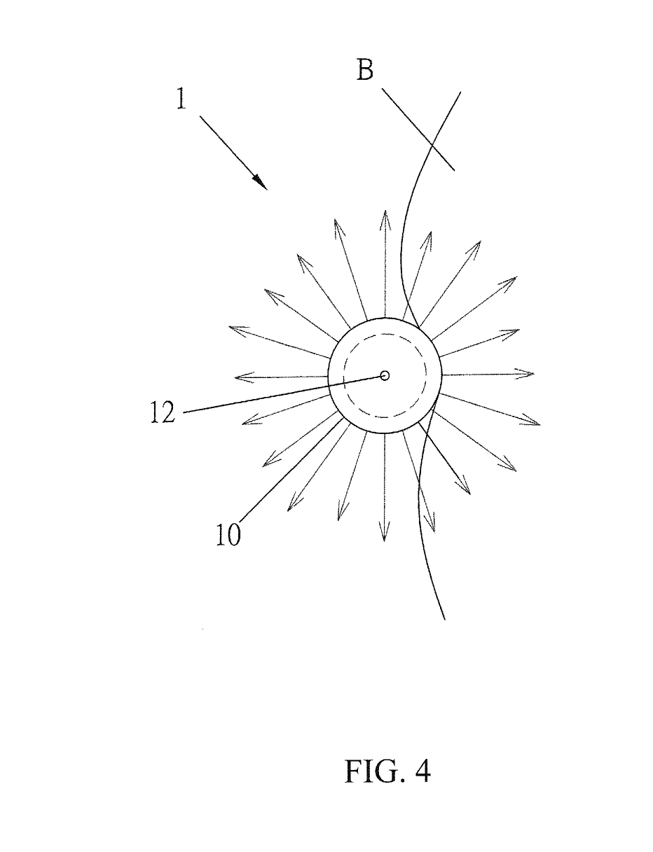

Referring to FIGS. 3 and 4, conditions of use of the two- and three-dimensional far infrared acupuncture device for therapy and rehabilitation 1 according to the first embodiment of the present invention are illustrated. To operate the two- and three-dimensional far infrared acupuncture device for therapy and rehabilitation 1 of the present invention, a user uses a hand to hold the handgrip section 21 and operate the control device 22 to set the temperature at a proper temperature level according to the need of a patient B. Afterwards, the portion that is enclosed by the tubular body 10 is placed to have a top side or a lateral side thereof contacting the body of the patient B, whereby the tubular body 10 emits far infrared light having a wavelength of 4-14 micrometers to stimulate the acupoints and organs of the human body for achieving an effect of therapy.

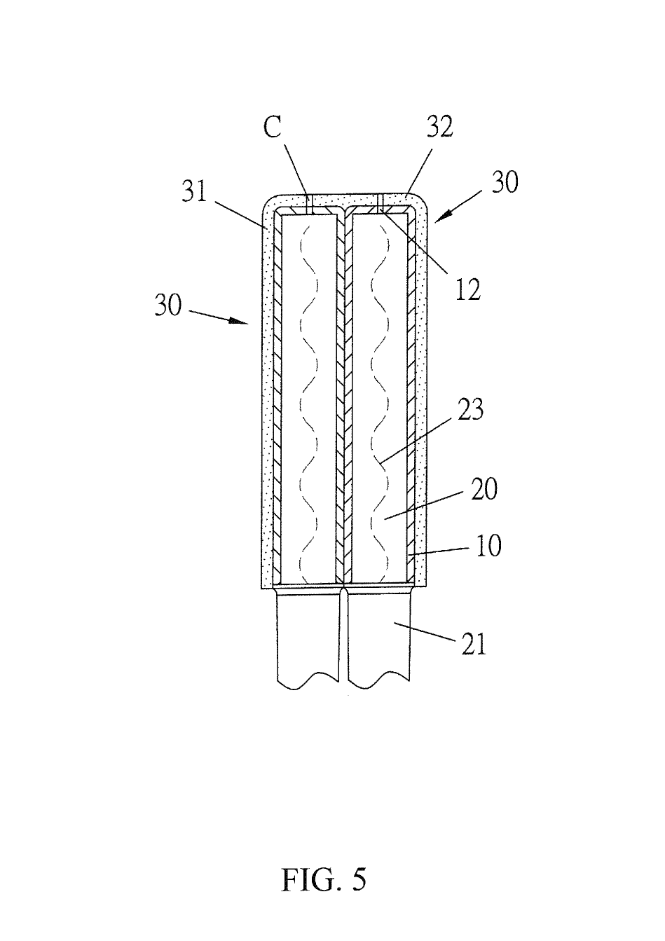

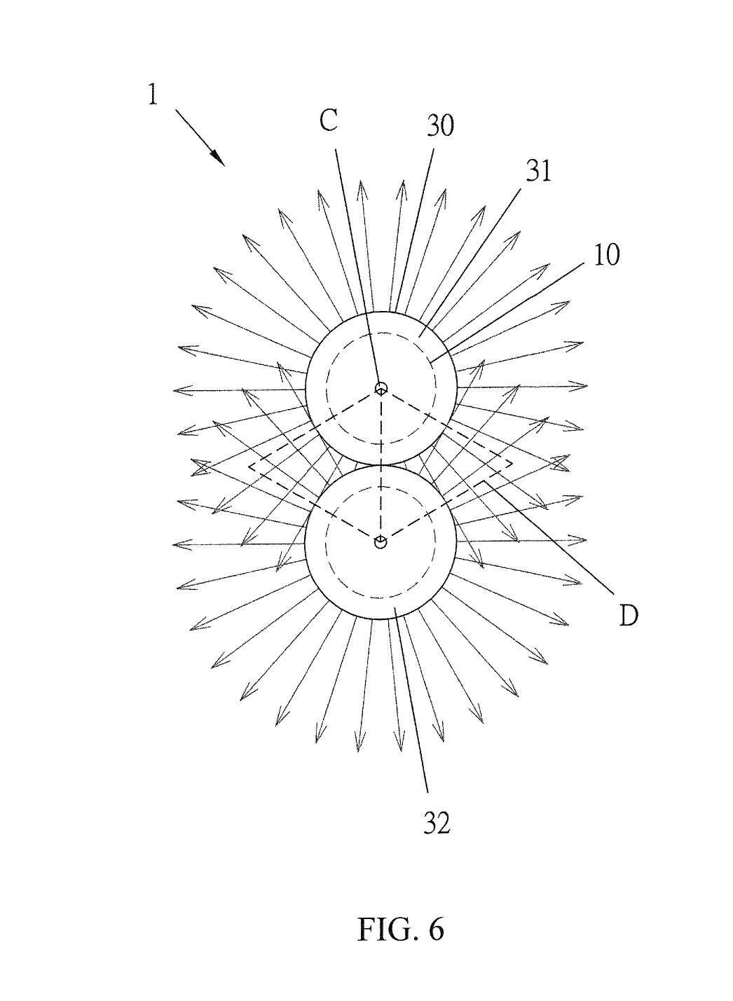

Referring to FIGS. 5 and 6, a schematic view, partly broken, showing a two- and three-dimensional far infrared acupuncture device for therapy and rehabilitation according to a second embodiment of the present invention, also designated at 1, and a condition of use thereof are illustrated.

The constituent components of the second embodiment are generally identical to those of the first embodiment and repeated description of the identical components will be omitted and only the differences thereof and the corresponding relationships thereof will be described.

The instant embodiment further comprises a connection enclosure unit 30, which comprises, formed therein, a plurality of first insertion holes 31 that are in communication with the outside and a plurality of second insertion holes 32 that are in communication with the outside. The first insertion holes 31 and the second insertion holes 32 are alternate with each other and are arranged side by side and parallel to each other. The first insertion holes 31 and the second insertion holes 32 are provided for receiving therein a plurality of tubular bodies 10.

Each of the first insertion holes 31 and each of the second insertion holes 32 has a top face in which a through aperture C is formed in such a way that the through aperture C corresponds to the ventilation hole 12 of the tubular body 10.

A plurality of electrical heating units 20 is respectively arranged in the tubular bodies 10 and is controlled by a control device 22 to set the temperature thereof within the range of 37-50 degrees Celsius so that the temperature is transmitted to each of the tubular bodies 10.

Since the tubular bodies 10 are arranged side by side, the tubular bodies 10 emit far infrared lights that intersect each other so that the intersection sites between the far infrared lights form at least two first equilateral-triangular far infrared light intersection zones D, by which the irradiation area and range and intensity of the far infrared lights are enhanced.

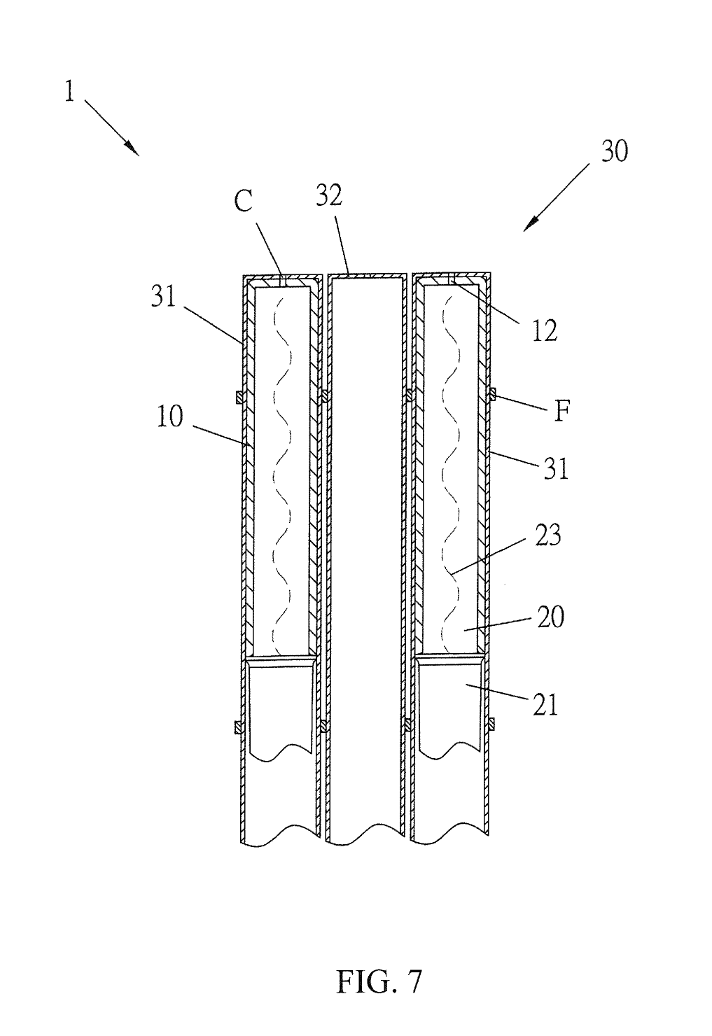

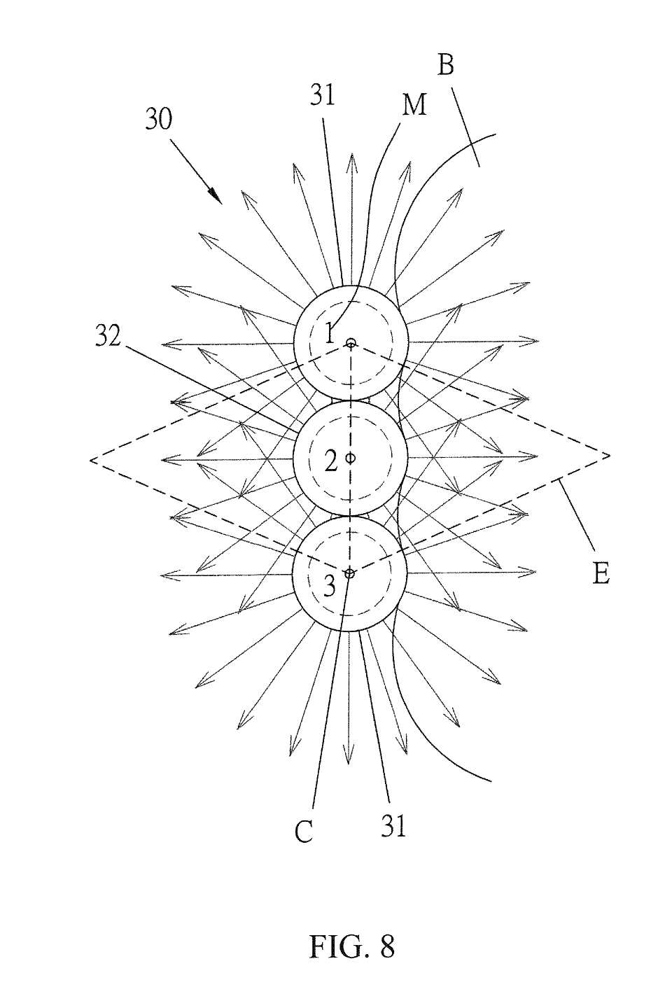

Referring to FIGS. 7 and 8, a schematic view, partly broken, showing a two- and three-dimensional far infrared acupuncture device for therapy and rehabilitation according to a third embodiment of the present invention, also designated at 1, and a condition of use thereof are illustrated.

The constituent components of the third embodiment are generally identical to those of the first and second embodiments and repeated description of the identical components will be omitted and only the differences thereof and the corresponding relationships thereof will be described.

In the instant embodiment, the tubular bodies 10 are respectively inserted into the first insertion holes 31, and the second insertion holes 32 are arranged between adjacent ones of the first insertion holes 31 so that the tubular bodies 10 each form, on two sides thereof, at least two second equilateral-triangular far infrared light intersection zones E.

Further, at least one flexible connection member F is arranged between each of the first insertion holes 31 and each of the second insertion holes 32 so that the first insertion holes 31 and the second insertion holes 32 can be arranged in a curved arrangement to form a loop configuration for surrounding the extremities or torso of a human body.

Further, each of the second insertion holes 32 may receive at least one metal bar (not shown) having a predetermined weight to insert therein to serve as a fixing point.

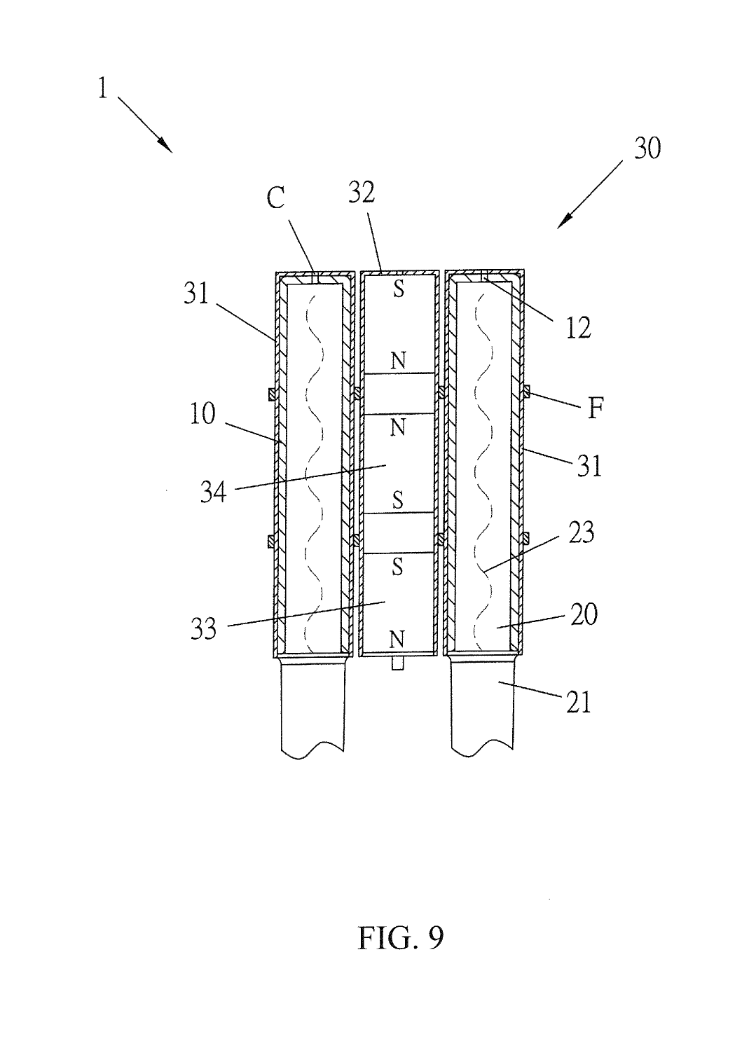

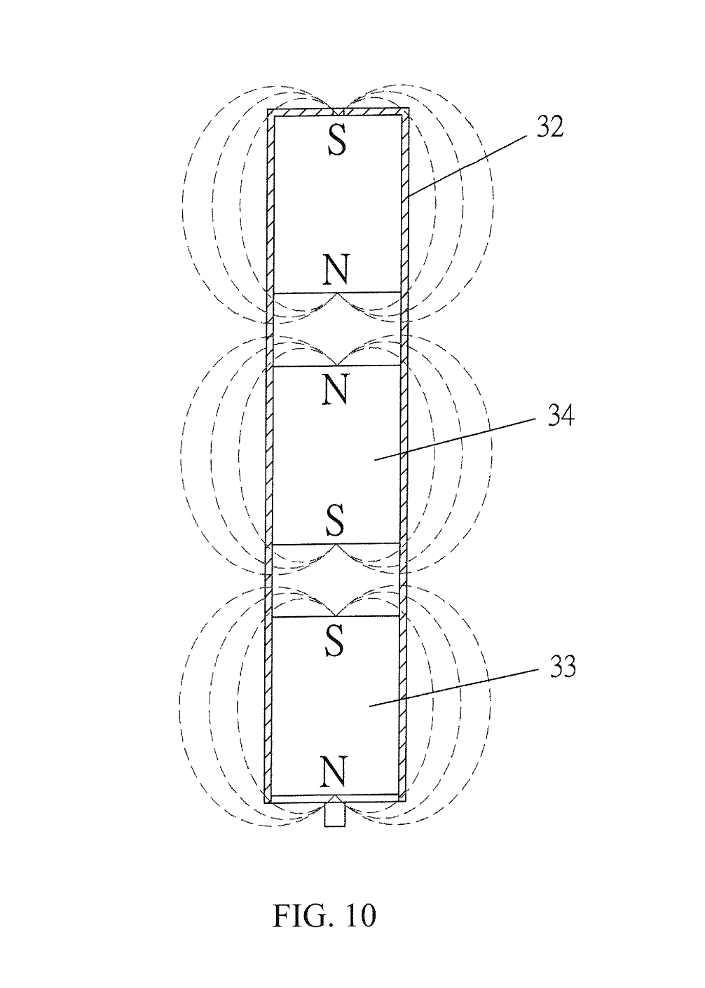

Referring to FIGS. 9 and 10, in a different embodiment, each of the second insertion holes 32 may receive at least one first magnet 33 and at least one second magnet 34 to position therein. The first magnet 33 has an end opposing an end of the second magnet 34 with the same magnetic polarity so as to induce a mutual expulsion effect resulting from the same polarity, whereby each first magnet 33 and each second magnet 34 induce magnetic fields therebetween in a segmented manner to allow the magnetic fields to cooperate with the far infrared lights to achieve an effect of relaxation or therapy.

In the second and third embodiments discussed above, the first insertion holes and the second insertion holes are sequentially provided with a number M or an English character (not shown in the drawings) to help make therapeutic records of the user (patient), whereby when a patient uses the two- and three-dimensional far infrared acupuncture device 1 for therapy and rehabilitation according to the present invention to perform therapy, doctors may record the first insertion holes 31 and the second insertion holes 32 of the connection enclosure unit 30 in which the two- and three-dimensional far infrared acupuncture devices 1 for therapy and rehabilitation are inserted so that in the next time of therapy, the two- and three-dimensional far infrared acupuncture devices 1 for therapy and rehabilitation can be installed according to the previous record in the first insertion holes 31 and the second insertion holes 32 to enhance the operation efficiency.

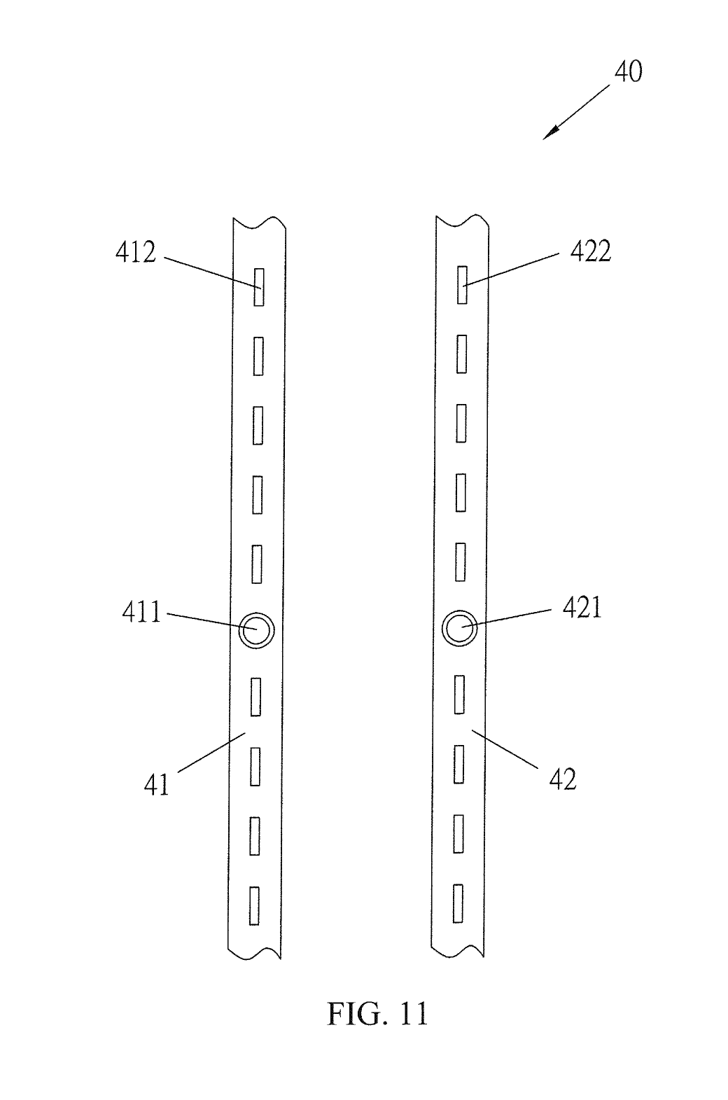

Referring to FIG. 11, a two- and three-dimensional far infrared acupuncture device for therapy and rehabilitation according to a fourth embodiment of the present invention, also designated at 1, is shown, wherein the tubular body 10 and the electrical heating unit 20 are of the same structures as those described in the previous embodiments so that repeated description will be omitted.

In the instant embodiment, the tubular body 10 and the electrical heating unit 20, after being assembled together, are mounted to a carrier unit 40 that comprises a first band 41 and a second band 42. The first band 41 and the second band 42 are each provided, at a predetermined locations, with a fixing hole 411, 421 and a plurality of insertion sections 412, 422. The fixing holes 411, 421 and the insertion sections 412, 422 are arranged in lines in a longitudinal direction and a two- and three-dimensional far infrared acupuncture device 1 for therapy and rehabilitation is inserted in a lateral direction into and retained in each of the insertion sections 412, 422.

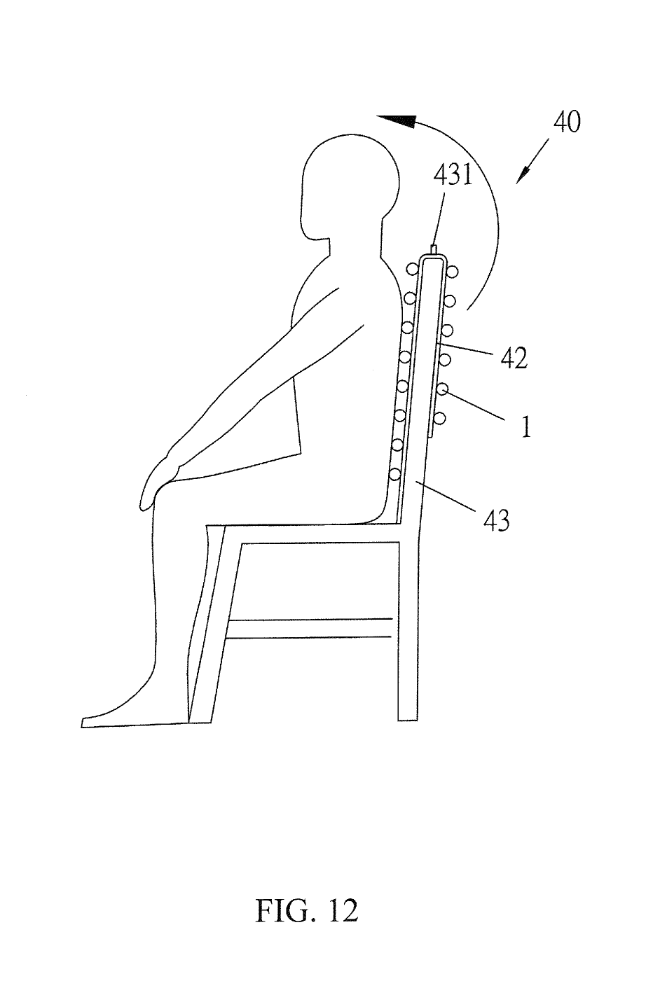

Referring to FIG. 12, the carrier unit 40 may further comprise a chair 43, which has a back having a top end on which two projections 431 are formed to mate and be coupled to the fixing holes 411, 421 of the first band 41 and the second band 42, whereby front and rear ends of each of the first band 41 and the second band 42 are respectively suspended on front and rear sides of the chair back. A user may sit on the chair 43 to use the two- and three-dimensional far infrared acupuncture devices 1 for therapy and rehabilitation according to the present invention to treat the back and the shoulders. The rear ends of the first band 41 and the second band 42 that are suspended on the projections 431 can be turned frontward so as to have the rear ends of the first band 41 and the second band 42 to be hung on the shoulders of the user for treating the chest or abdomen of the user.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A two- and three-dimensional far infrared acupuncture device for therapy and rehabilitation, comprising:
at least one tubular body, which comprises a far infrared ceramic material, the tubular body having two end respectively forming an opening and a ventilation hole and comprising an accommodation space formed between the opening and the ventilation hole; and
at least one electrical heating unit, which has an end forming a handgrip section, the handgrip section comprising a control device mounted thereto, the electrical heating unit having an end that is opposite to the handgrip section and is insertable through the opening into the accommodation space of the tubular body, the control device controlling temperature of the electrical heating unit within a range of 37-50 degrees Celsius so that the temperature is transmitted to the tubular body, whereby the tubular body emits far infrared light having a wavelength of 4-14 micrometers to stimulate acupoints and organs of a human body for achieving an effect of therapy.

2. The two- and three-dimensional far infrared acupuncture device for therapy and rehabilitation according to claim 1, wherein the electrical heating unit comprises at least one nichrome wire arranged therein and electrically connected with the control device.

3. A two- and three-dimensional far infrared acupuncture device for therapy and rehabilitation, comprising:
a connection enclosure unit, which comprises, formed therein, a plurality of first insertion holes that are in communication with the outside and a plurality of second insertion holes that are in communication with the outside, the first insertion holes and the second insertion holes being alternate with each other and arranged side by side and parallel to each other;
a plurality of tubular bodies, each of which comprises a far infrared ceramic material, each of the tubular bodies having two end respectively forming an opening and a ventilation hole and comprising an accommodation space formed between the opening and the ventilation hole, the tubular bodies being insertable into the first insertion holes and the second insertion holes; and
a plurality of electrical heating units, each of which has an end forming a handgrip section, the handgrip section comprising a control device mounted thereto, each of the electrical heating units having an end that is opposite to the handgrip section and is insertable through the opening into a corresponding one of the accommodation spaces, the control device controlling temperature of the electrical heating unit within a range of 37-50 degrees Celsius so that the temperature is transmitted to the corresponding one of the tubular bodies, whereby the tubular bodies emit far infrared lights having a wavelength of 4-14 micrometers and intersecting each other so as form at least two first equilateral-triangular far infrared light intersection zones.

4. The two- and three-dimensional far infrared acupuncture device for therapy and rehabilitation according to claim 3, wherein the tubular bodies are inserted in the first insertion holes and the second insertion holes are arranged between the first insertion holes so that the tubular bodies each form, on two sides thereof, at least two second equilateral-triangular far infrared light intersection zones.

5. The two- and three-dimensional far infrared acupuncture device for therapy and rehabilitation according to claim 3, wherein at least one flexible connection member is arranged between each of the first insertion holes and each of the second insertion holes.

6. The two- and three-dimensional far infrared acupuncture device for therapy and rehabilitation according to claim 3, wherein each of the second insertion holes receives at least one at least one metal bar having a predetermined weight to insert therein to serve as a fixing point.

7. The two- and three-dimensional far infrared acupuncture device for therapy and rehabilitation according to claim 3, wherein each of the second insertion holes receives at least two magnetic elements to insert therein, magnetic polarities of the magnetic elements being arranged to have the magnetic elements mutually expel each other to form a plurality of magnetic fields.

8. The two- and three-dimensional far infrared acupuncture device for therapy and rehabilitation according to claim 3, wherein the electrical heating units each comprise at least one nichrome wire arranged therein and electrically connected with the control device.

9. The two- and three-dimensional far infrared acupuncture device for therapy and rehabilitation according to claim 3, wherein the first insertion holes and the second insertion holes are sequentially provided with a number or an English character.

10. The two- and three-dimensional far infrared acupuncture device for therapy and rehabilitation according to claim 3, wherein the first insertion holes and the second insertion holes each have a top face in which a through aperture is formed, the through aperture corresponding to the ventilation hole of the tubular body.
